(19) United States
(12) Patent Application Publication
(10) Pub. No.: US 2024/0194305 A1
(43) Pub. Date: Jun. 13, 2024

(54) MACHINE LEARNING ENABLED TECHNIQUES FOR MATERIAL DESIGN AND ULTRA-INCOMPRESSIBLE TERNARY COMPOUNDS DERIVED THEREWITH

(71) Applicant: The Regents of the University of California, Oakland, CA (US)

(72) Inventors: Shyue Ping Ong, San Diego, CA (US); Jian Luo, San Diego, CA (US); Yunxing Zuo, San Diego, CA (US); Mingde Qin, San Diego, CA (US); Chi Chen, San Diego, CA (US)

(21) Appl. No.: 18/555,664

(22) PCT Filed: Apr. 15, 2022
(86) PCT No.: PCT/US22/25085

§ 371 Date: Oct. 16, 2023

Related U.S. Application Data
(60) Provisional application No. 63/175,697, filed on Apr. 16, 2021.

(51) Int. Cl. G16C 20/70; G06N 3/04

(52) U.S. Cl. CPC G16C 20/70; G06N 3/04

(57) ABSTRACT

A method for machine learning enabled material design may include applying a first machine learning model trained to generate an equilibrium crystal structure corresponding a crystal structure generated, for example, by performing an elemental substation. The first machine learning model may generate the equilibrium crystal structure by iteratively searching a solution space including possible variations of the crystal structure for a variation having a minimum formation energy. The searching may be constrained to variations having a same symmetry as the crystal structure. Properties of the crystal structure may be determined, for example, by applying a second machine learning to the equilibrium crystal structure. The crystal structure may be identified as a candidate for synthesis based the properties of the crystal structure, such as an above-threshold elastic modulus corresponding to an ultra-incompressibility. Various materials identified using this method and related systems and computer program products are also provided.

### CROSS REFERENCE TO RELATED APPLICATION

This application claims priority to U.S. Provisional Application No. 63/175,697, entitled “NEW ULTRA-INCOMPRESSIBLE TERNARY COMPOUNDS DISCOVERED BY MACHINE LEARNING MODELS AND HIGH-ENERGY BALL MILL AND SPARK PLASMA SINTERING TECHNIQUES” and filed on Apr. 16, 2021, the disclosure of which is incorporated herein by reference in its entirety.

### STATEMENT OF GOVERNMENT SUPPORT

This invention was made with government support under DMR2011967 and ACI1548562 awarded by the National Science Foundation, and under DE-AC02-05CH11231 awarded by the Department of Energy. The government has certain rights in the invention.

### TECHNICAL FIELD

The subject matter described herein relates generally to materials science and more specifically to machine learning enabled techniques for material design and new materials derived therewith.

### BACKGROUND

Materials science is an interdisciplinary field that includes the discovery and design of new materials including, for example, ceramics, polymers, semiconductors, magnets, biomaterials, nanomaterials, and/or the like. The endeavor to discover and design a new material may include an exploration of the relationship the structure of the material, the methods for processing the material, and the properties exhibited by the material. For example, the structure of the material may be evaluated on an atomic scale using a variety of techniques such as diffraction (e.g., with X-rays, electrons, or neutrons), spectroscopy (e.g., Raman spectroscopy, energy-dispersive spectroscopy), thermal analysis, chemical assays (e.g., chromatography), and microscopy (e.g., with an electron microscope). The utility of the material, including its suitability for certain applications, may be contingent upon the properties exhibited by the material. Examples of material properties include mechanical properties, chemical properties, electrical properties, thermal properties, optical properties, magnetic properties, and/or the like.

### SUMMARY

Systems, methods, and articles of manufacture, including computer program products, are provided for machine learning enabled material design. In some example embodiments, there is provided a system that includes at least one processor and at least one memory. The at least one memory may include program code that provides operations when executed by the at least one processor. The operations may include: applying a first machine learning model trained to generate, based at least on a first crystal structure, an equilibrium crystal structure corresponding the first crystal structure, the first machine learning model generating the equilibrium crystal structure by at least iteratively searching a solution space including a plurality of possible variations of the first crystal structure for a variation of the first crystal structure having a minimum formation energy; determining, based at least on the equilibrium crystal structure, one or more properties of the first crystal structure; and identifying, based at least on the one or more properties of the first crystal structure, the first crystal structure as a candidate for synthesis.

In some variations, one or more features disclosed herein including the following features can optionally be included in any feasible combination. The first machine learning model comprises a Bayesian optimization (BO) model.

In some variations, the searching of the solution space may be constrained to variations of the first crystal structure having a same symmetry as the first crystal structure.

In some variations, the searching of the solution space may be constrained based on a symmetry of a lattice of the first crystal structure.

In some variations, the searching of the solution space may be constrained based on a Wyckoff position of each atom comprising the first crystal structure.

In some variations, each variation of the plurality of possible variations of the first crystal structure may include at least one change to a lattice parameter or an atomic coordinate of the first crystal structure.

In some variations, the first machine learning model may generate the equilibrium crystal structure by at least searching the solution space to identify a first variation of the first crystal structure and determining a first formation energy of the first variation of the first crystal structure.

In some variations, the first machine learning model may further generate the equilibrium crystal structure by at least searching the solution space to identify a second variation of the first crystal structure, determining a second formation energy of the second variation of the first crystal structure, and in response to the second formation energy being less than the first formation energy, searching, based at least on the second variation of the first crystal structure, the solution space to identify a third variation of the first crystal structure.

In some variations, the searching of the solution space may include exploiting an explored portion the search space by at least identifying a sample having an above-threshold predicted mean as the third variation of the first crystal structure.

In some variations, the first formation energy and the second formation energy may be determined by applying a second machine learning model.

In some variations, the one or more properties may be determined by applying a second machine learning model trained to determine the one or more properties.

In some variations, the second machine learning model may include a graph neural network in which atoms in a crystal structure are represented as nodes and bonds in the crystal structure as edges.

In some variations, the one or more properties may include at least one of a mechanical property, chemical property, thermal property, optical property, or magnetic property of the equilibrium crystal structure.

In some variations, the operations may further include generating, based at least on a second crystal structure, the first crystal structure.

In some variations, the first crystal structure may be generated by at least substituting a first element of the second crystal structure with a second element.

In some variations, the first crystal structure may be identified as the candidate for synthesis based at least on the first crystal structure exhibiting an above-threshold elastic modulus.

In some variations, the first crystal structure may be identified as the candidate for synthesis by in-situ reactive spark plasma sintering.

In another aspect, there is provided a method for machine learning enabled material design. The method may include: applying a first machine learning model trained to generate, based at least on a first crystal structure, an equilibrium crystal structure corresponding the first crystal structure, the first machine learning model generating the equilibrium crystal structure by at least iteratively searching a solution space including a plurality of possible variations of the first crystal structure for a variation of the first crystal structure having a minimum formation energy; determining, based at least on the equilibrium crystal structure, one or more properties of the first crystal structure; and identifying, based at least on the one or more properties of the first crystal structure, the first crystal structure as a candidate for synthesis.

In some variations, one or more features disclosed herein including the following features can optionally be included in any feasible combination.

The first machine learning model comprises a Bayesian optimization (BO) model.

In some variations, the searching of the solution space may be constrained to variations of the first crystal structure having a same symmetry as the first crystal structure.

In some variations, the searching of the solution space may be constrained based on a symmetry of a lattice of the first crystal structure.

In some variations, the searching of the solution space may be constrained based on a Wyckoff position of each atom comprising the first crystal structure.

In some variations, each variation of the plurality of possible variations of the first crystal structure may include at least one change to a lattice parameter or an atomic coordinate of the first crystal structure.

In some variations, the first machine learning model may generate the equilibrium crystal structure by at least searching the solution space to identify a first variation of the first crystal structure and determining a first formation energy of the first variation of the first crystal structure.

In some variations, the first machine learning model may further generate the equilibrium crystal structure by at least searching the solution space to identify a second variation of the first crystal structure, determining a second formation energy of the second variation of the first crystal structure, and in response to the second formation energy being less than the first formation energy, searching, based at least on the second variation of the first crystal structure, the solution space to identify a third variation of the first crystal structure.

In some variations, the searching of the solution space may include exploiting an explored portion the search space by at least identifying a sample having an above-threshold predicted mean as the third variation of the first crystal structure.

In some variations, the first formation energy and the second formation energy may be determined by applying a second machine learning model.

In some variations, the one or more properties may be determined by applying a second machine learning model trained to determine the one or more properties.

In some variations, the second machine learning model may include a graph neural network in which atoms in a crystal structure are represented as nodes and bonds in the crystal structure as edges.

In some variations, the one or more properties may include at least one of a mechanical property, chemical property, thermal property, optical property, or magnetic property of the equilibrium crystal structure.

In some variations, the method may further include generating, based at least on a second crystal structure, the first crystal structure.

In some variations, the first crystal structure may be generated by at least substituting a first element of the second crystal structure with a second element.

In some variations, the first crystal structure may be identified as the candidate for synthesis based at least on the first crystal structure exhibiting an above-threshold elastic modulus.

In some variations, the first crystal structure may be identified as the candidate for synthesis by in-situ reactive spark plasma sintering.

In another aspect, there is provided a computer program product including a non-transitory computer readable medium storing instructions. The instructions may cause operations may executed by at least one data processor. The operations may include: applying a first machine learning model trained to generate, based at least on a first crystal structure, an equilibrium crystal structure corresponding the first crystal structure, the first machine learning model generating the equilibrium crystal structure by at least iteratively searching a solution space including a plurality of possible variations of the first crystal structure for a variation of the first crystal structure having a minimum formation energy; determining, based at least on the equilibrium crystal structure, one or more properties of the first crystal structure; and identifying, based at least on the one or more properties of the first crystal structure, the first crystal structure as a candidate for synthesis.

In another aspect, there is provided an apparatus that comprises: means applying a first machine learning model trained to generate, based at least on a first crystal structure, an equilibrium crystal structure corresponding the first crystal structure, the first machine learning model generating the equilibrium crystal structure by at least iteratively searching a solution space including a plurality of possible variations of the first crystal structure for a variation of the first crystal structure having a minimum formation energy; means for determining, based at least on the equilibrium crystal structure, one or more properties of the first crystal structure; and means for identifying, based at least on the one or more properties of the first crystal structure, the first crystal structure as a candidate for synthesis.

In another aspect, there is provided a crystalline form of a compound having a chemical formula of ReWB and characterized by an X-ray powder diffraction pattern comprising peaks at 41.29, 32.35, 38.51, 73.47, and 52.31 (±0.1 degrees 2θ).

In some variations, the X-ray powder diffraction pattern may further include peaks at 71.54, 51.14, 22.71, 66.80, and 72.49 (±0.1 degrees 2θ).

In another aspect, there is provided a crystalline form of a compound having a chemical formula of (Mo_{0.5}W_{0.5})₂B, and characterized by an X-ray powder diffraction pattern comprising peaks at 40.97, 32.18, 37.96, 50.59, and 70.60 (±0.1 degrees 2θ).

In some variations, the X-ray powder diffraction pattern may further include peaks at 72.89, 51.96, 22.61, 66.13, and 71.98 (±0.1 degrees 2θ).

In another aspect, there is provided a crystalline form of a compound having a chemical formula of (Mo_{0.5}W_{0.5})₂C and characterized by an X-ray powder diffraction pattern comprising peaks at 39.31, 39.45, 37.90, 69.47, and 52.05 (±0.1 degrees 2θ).

In some variations, the X-ray powder diffraction pattern may further include peaks at 34.26, 74.67, 34.38, 61.58, and 75.40 (±0.1 degrees 2θ).

In another aspect, there is provided a crystalline form of a compound having a chemical formula of MoWC₂ and characterized by an X-ray powder diffraction pattern comprising peaks at 35.65, 48.35, 31.58, 73.15, and 64.02 (±0.1 degrees 2θ).

In some variations, the X-ray powder diffraction pattern may further include peaks at 77.30, 75.47, and 65.96 (±0.1 degrees 2θ).

Implementations of the current subject matter can include, but are not limited to, systems and methods consistent including one or more features are described as well as articles that comprise a tangibly embodied machine-readable medium operable to cause one or more machines (e.g., computers, etc.) to result in operations described herein. Similarly, computer systems are also described that may include one or more processors and one or more memories coupled to the one or more processors. A memory, which can include a computer-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to machine learning enabled material design and ultra-incompressible ternary compounds derived therewith, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a material design system, in accordance with some example embodiments;

FIG. 2 depicts a schematic diagram illustrating an example of the Bayesian optimization with symmetry relaxation (BOWSR) algorithm, in accordance with some example embodiments;

FIG. 3 depicts a flowchart illustrating an example of a process for material discovery that leverages the Bayesian optimization with symmetry relaxation (BOWSR) algorithm and the MatErials Graph Network (MEGNet) formation energy model, in accordance with some example embodiments;

FIG. 4 depicts a schematic diagram illustrating an example of a specimen preparation process, in accordance with some example embodiments;

FIG. 5 depicts examples of new materials identified by applying the Bayesian optimization with symmetry relaxation (BOWSR) algorithm and the MatErials Graph Network (MEGNet) formation energy model, in accordance with some example embodiments;

FIG. 6 depicts experimental measurements and theoretical predictions of the mechanical properties of the examples of new materials identified by applying the Bayesian optimization with symmetry relaxation (BOWSR) algorithm and the MatErials Graph Network (MEGNet) formation energy model, in accordance with some example embodiments;

FIG. 7 depicts a graph illustrating the measured X-ray power diffraction (XRD) patterns of the new materials identified by applying the Bayesian optimization with symmetry relaxation (BOWSR) algorithm and the MatErials Graph Network (MEGNet) formation energy model, in accordance with some example embodiments;

FIG. 8 depicts the convergence of the Bayesian optimization with symmetry relaxation (BOWSR) algorithm using the MatErials Graph Network (MEGNet) formation energy model for various structures in a dataset used for material property prediction, in accordance with some example embodiments;

FIG. 9 depicts a statistical distribution of a dataset used for material property prediction, in accordance with some example embodiments;

FIG. 10 depicts a graph illustrating the mean absolute errors (MAEs) of predictions made using the MatErials Graph Network (MEGNet) compared to density functional theory (DFT) ground state calculations in formation energies using unrelaxed crystal structures and crystal structures relaxed using the Bayesian optimization with symmetry relaxation (BOWSR) algorithm grouped by structure prototype, in accordance with some example embodiments;

FIG. 11 depicts graphs illustrating the sensitivity of the Bayesian optimization with symmetry relaxation (BOWSR) algorithm to the accuracy of the energy model, in accordance with some example embodiments;

FIG. 12 depicts a graph illustrating the measured X-ray power diffraction (XRD) patterns of the ReOsB specimen, in accordance with some example embodiments;

FIG. 13 depicts a graph illustrating the measured X-ray power diffraction (XRD) patterns of the ReOsB₂ specimen, in accordance with some example embodiments;

FIG. 14 depicts a graph illustrating the measured X-ray power diffraction (XRD) patterns of the Re₁₃WB₉ specimen, in accordance with some example embodiments;

FIG. 15 depicts a graph illustrating the measured X-ray power diffraction (XRD) patterns of the OSWB specimen, in accordance with some example embodiments;

FIG. 16 depicts a graph illustrating the measured X-ray power diffraction (XRD) patterns of the Re₆W₇B₈ specimen, in accordance with some example embodiments;

FIG. 17 depicts graphs illustrating the measured X-ray power diffraction (XRD) patterns of the ReW₂B₂ specimen, in accordance with some example embodiments;

FIG. 18 depicts a graph illustrating the mean absolute errors (MAEs) of predictions made using the MatErials Graph Network (MEGNet) compared to density functional theory (DFT) ground state calculations in formation energies of crystal structures relaxed using the Bayesian optimization with symmetry relaxation (BOWSR) algorithm with and without symmetry constraints, in accordance with some example embodiments;

FIG. 19 depicts a flowchart illustrating an example of a process for machine learning enabled material design, in accordance with some example embodiments;

FIG. 20 depicts a block diagram illustrating an example of a computing system, in accordance with some example embodiments;

FIG. 21A depicts the characteristics of ReWB, in accordance with some example embodiments;

FIG. 21B depicts the characteristics of (Mo_{0.5}W_{0.5})₂B, in accordance with some example embodiments;

FIG. 21C depicts the characteristics of (Mo_{0.5}W_{0.5})₂C, in accordance with some example embodiments; and

FIG. 21D depicts the characteristics of MoWC₂, in accordance with some example embodiments.

When practical, similar reference numbers denote similar structures, features, or elements.

### DETAILED DESCRIPTION

The accurate prediction of novel stable crystals and their properties is a fundamental goal in computation-guided materials discovery. While ab initio approaches such as density functional theory (DFT) have been phenomenally successful in this regard, their high computational cost and poor scalability have limited the broad application across vast chemical and structural spaces. As a result, high-throughput density functional theory based screening has been mostly performed on ˜O(100-1000) crystals with relatively small unit cells. To circumvent the limitation associated with density functional theory based screening, machine learning (ML) has emerged as a new paradigm for developing efficient surrogate models for predicting materials properties at scale. Such machine learning models are usually trained on large databases of materials properties in order to learn the relationship between input chemical and/or structural features and target properties (e.g., formation energies, band gaps, elastic moduli, and/or the like).

Machine learning models that have been trained on structural as well as chemical features may be capable of distinguishing between polymorphs and be universally applied in materials discovery across diverse crystal structures. In particular, graph neural networks, where atoms and bonds in crystal structures are represented as nodes and edges in a mathematical graph, may provide highly accurate results in predicting a broad range of energetic, electronic, and mechanical properties. Nevertheless, the requirement for equilibrium crystal structures to be provided as inputs remains a critical bottleneck in the application of structure-based machine learning models for materials discovery. Equilibrium crystal structures are obtained by “relaxing” initial input structures along their potential energy surfaces, typically using expensive density functional theory (DFT) based calculations. Thus, while machine learning (ML) models utilizing structure-based features can provide an efficient means for accurate property predictions across diverse chemical spaces, the reliance on density functional theory (DFT) calculations to obtain equilibrium crystal structures continues to limit machine learning-based exploration to either known crystals or a small number of hypothetical crystals.

In some example embodiments, a Bayesian optimization with symmetry relaxation (BOWSR) algorithm may be applied to obtain equilibrium crystal structures for subsequent property prediction using, for example, a machine learning based property prediction model such as a graph based deep learning energy model. This approach avoids conventional density functional theory (DFT) calculations but still provides highly accurate equilibrium crystal structures as inputs for the machine learning based property prediction model. As such, application of the Bayesian optimization with symmetry relaxation (BOWSR) algorithm significantly improves the accuracy of the machine learning based property prediction model. For example, when the Bayesian optimization with symmetry relaxation (BOWSR) algorithm is applied to determine the equilibrium state of a hypothetical crystal structure, the machine learning based property prediction model (e.g., the graph based deep learning energy model) may yield highly accurate formation energies and elastic moduli for the hypothetical crystal structure.

In some example embodiments, a combination of the Bayesian optimization with symmetry relaxation (BOWSR) algorithm and the machine learning based property prediction model (e.g., the graph based deep learning energy model) may be applied to screen a large quantity of candidate crystal structures to identify those exhibiting certain exceptional (or other desirable) properties. For example, a combination of the Bayesian optimization with symmetry relaxation (BOWSR) algorithm and the machine learning based property prediction model (e.g., the graph based deep learning energy model) may be applied to screen approximately 400,000 transition metal borides and carbides to identify materials exhibiting exceptional mechanical properties such as ultra-incompressibility. In doing so, a variety of novel ultra-incompressible hard materials, including MoWC₂ (P6₃/mmc) and ReWB (Pca2₁), are identified and then synthesized, for example, via in-situ reactive spark plasma sintering.

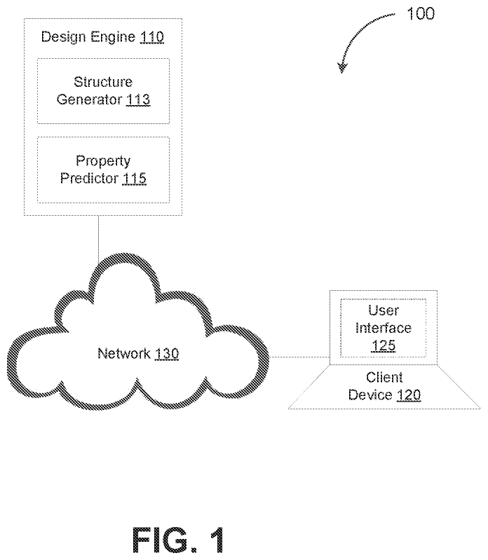

FIG. 1 depicts a system diagram illustrating an example of a material design system 100, in accordance with some example embodiments. Referring to FIG. 1, the material design system 100 may include a design engine 110 and a client device 120. As shown in FIG. 1, the design engine 110 and the client device 120 may be communicatively coupled via a network 130. The client device 120 may be a processor-based device including, for example, a workstation, a desktop computer, a laptop computer, a smartphone, a tablet computer, a wearable apparatus, and/or the like. The network 130 may be a wired network and/or a wireless network including, for example, a local area network (LAN), a virtual local area network (VLAN), a wide area network (WAN), a public land mobile network (PLMN), the Internet, and/or the like.

Referring again to FIG. 1, the design engine 110 may include a structure generator 113 configured to generate, for each input crystal structure, a corresponding equilibrium crystal structure. The design engine 110 may further include a property predictor 115 configured to determine one or more properties for each equilibrium crystal structure generated by the structure generator 113. In some cases, one or more of the input crystal structures determined to exhibit certain exceptional (or other desirable) properties may be provided for display, for example, in a user interface 125 of the client device 120. For example, the user interface 125 may display one or more of the input crystal structures that exhibit an ultra-incompressibility. Moreover, in some cases, the input crystal structures determined to exhibit exceptional (or other desirable) properties may be identified as candidate for synthesis (e.g., via in-situ reactive spark plasma sintering (SPS) and/or the like) and further evaluation.

In some example embodiments, the structure generator 113 may be configured to apply a Bayesian optimization with symmetry relaxation (BOWSR) algorithm in order to “relax” an input crystal structure along its potential energy surfaces and generate a corresponding equilibrium crystal structure. Meanwhile, to determine one or more properties of the equilibrium crystal structure, the property predictor 115 may apply a machine learning based property prediction model, such as a graph based deep learning energy model, in which atoms and bonds in crystal structures are represented as nodes and edges in a mathematical graph.

Bayesian optimization (BO) is an adaptive strategy for the global optimization of a function. In the case of crystal structure relaxation, the function to be optimized is the potential energy surface, which expresses the energy of the crystal structure as a function of the lattice parameters and atomic coordinates. In the Bayesian optimization with symmetry relaxation (BOWSR) algorithm, the symmetry (space group) of the lattice and the Wyckoff positions of the atoms are constrained during the relaxation process. For example, an equilibrium crystal structure may be achieved by varying only the independent lattice parameters and atomic coordinates of the input crystal structure. The goal of Bayesian optimization is then to minimize the mapping shown as Equations (1) and (2) below.

$$x := \{ a , b , c , α , β , γ , \vec{{c}_{1}} , \vec{{c}_{2}} , … \} \qquad ( 1 )$$

$${x}_{o p t} = {arg min}_{x} U ( x ) , U : {R}^{n} \to R \qquad ( 2 )$$

wherein {a, b, c, α, β, γ} denote the independent lattice parameters of a P1 crystal, {\vec{c₁}, \vec{c₂}, . . . } denote the atomic positions of the P1 crystal, and U (x) denotes the energy of the system.

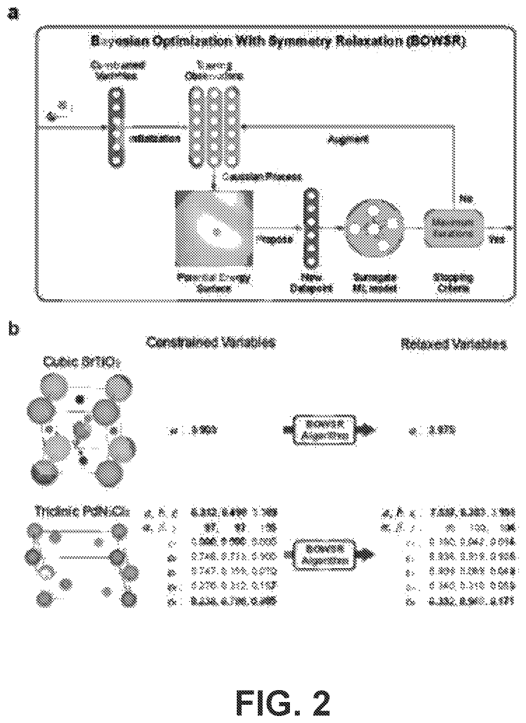

FIG. 2 depicts a schematic diagram illustrating an example of the Bayesian optimization with symmetry relaxation (BOWSR) algorithm, in accordance with some example embodiments. Referring to FIG. 2(a), the Bayesian optimization with symmetry relaxation (BOWSR) algorithm parameterizes each crystal based on the independent lattice parameters and coordinates based on its space group. The potential energy surface is then approximated by initializing a set of training parameters and energies from a machine learning based energy model (e.g., the graph based deep learning energy model applied by the property predictor 115). Bayesian optimization is then used to iteratively propose lower energy geometries based on prior observations. FIG. 2(b) shows two examples of the geometry parameterization for cubic perovskite SrTiO; and triclinic PdN₂Cl₂. For the high symmetry cubic perovskite, the a lattice parameter is the only independent parameter, and all atoms are in special Wyckoff positions with constrained crystal coordinates. Contrastingly, for the triclinic crystal, all six lattice parameters and all atomic coordinates may be independent parameters.

The convergence accuracy and speed of the Bayesian optimization with symmetry relaxation (BOWSR) algorithm may be determined by the energy evaluator U(.). It should be appreciated that the energy evaluator U(.) may be implemented as a variety of computational models including, for example, ab initio methods, empirical potentials, surrogate machine learning models, and/or the like. In some example embodiments, the energy evaluator U(.) may be implemented as the graph based deep learning energy model applied by the property predictor 115. One example of a graph based deep learning energy model is a MatErials Graph Network (MEGNet) formation energy model trained, for example, based the formation energies of 133,420 Materials Project crystals determined by applying density functional theory (DFT) based calculations. This MEGNet formation energy model exhibits a cross-validated mean absolute error (MAE) of 26 meV atom⁻¹, which is among the best accuracy among general machine learning models thus far.

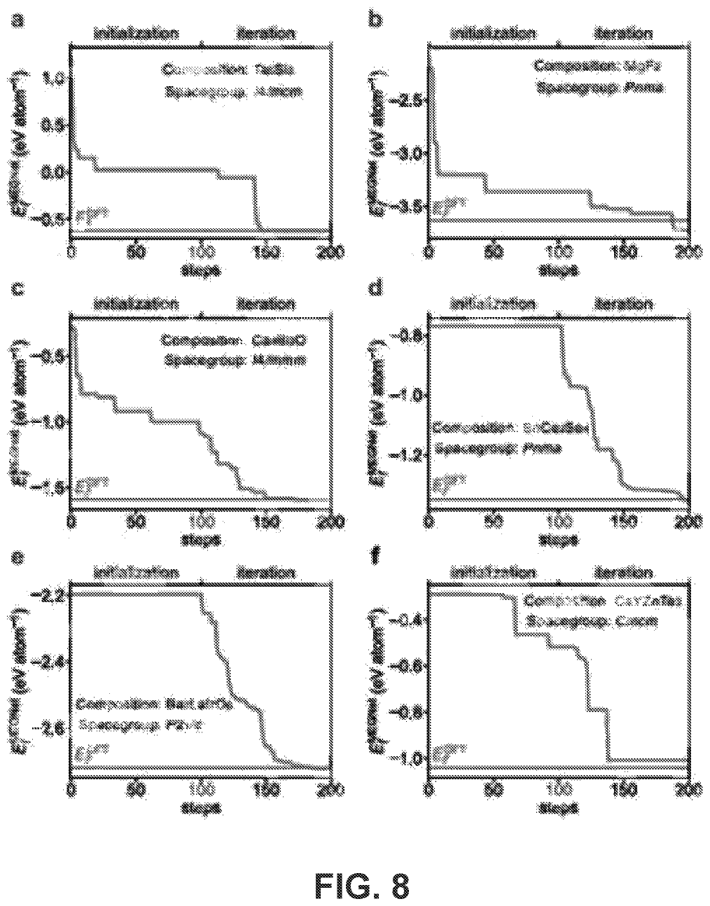

FIG. 8 depicts examples of the convergence of the Bayesian optimization with symmetry relaxation (BOWSR) algorithm using the MatErials Graph Network (MEGNet) formation energy model for various structures in a dataset used for material property prediction, in accordance with some example embodiments. Referring to FIG. 8, the structures in the dataset includes two binary, two ternary, and two quaternary crystals obtained from elemental substitution in commonly occurring structure prototypes. The structure prototypes include a, Ta₅Si₃, b, MgF₂, c, Ca4Bi2O, d, SnCe2Se4, e, Ba₂LaIrO₆, f, CsYZnTe₃ are Cr5B3 (ICSD #27124), Sr₂Si (ICSD #422), K₂NiF₄ (ICSD #15576), CaFe₂O₄ (ICSD #28177), La₂ZnIrO6 (ICSD #75596), and KZrCuS₃ (ICSD #80624), respectively. All structures were relaxed via the Bayesian optimization with symmetry relaxation (BOWSR) algorithm using the default number of initialization samples (100) and iterations (100).

Elemental substitution may be a chemically intuitive approach to deriving potential new compounds. For example, the rock salt lithium chloride (LiCl) can be derived from the rock salt sodium chloride (NaCl) by substituting sodium (Na) for the chemically similar element lithium (Li). For candidate crystal structures generated via elemental substitution, machine learning based property prediction may yield more accurate results (e.g., for formation energies and elastic moduli (bulk and shear moduli)) when applied to equilibrium crystal structures that have been relaxed using the Bayesian optimization with symmetry relaxation (BOWSR) algorithm.

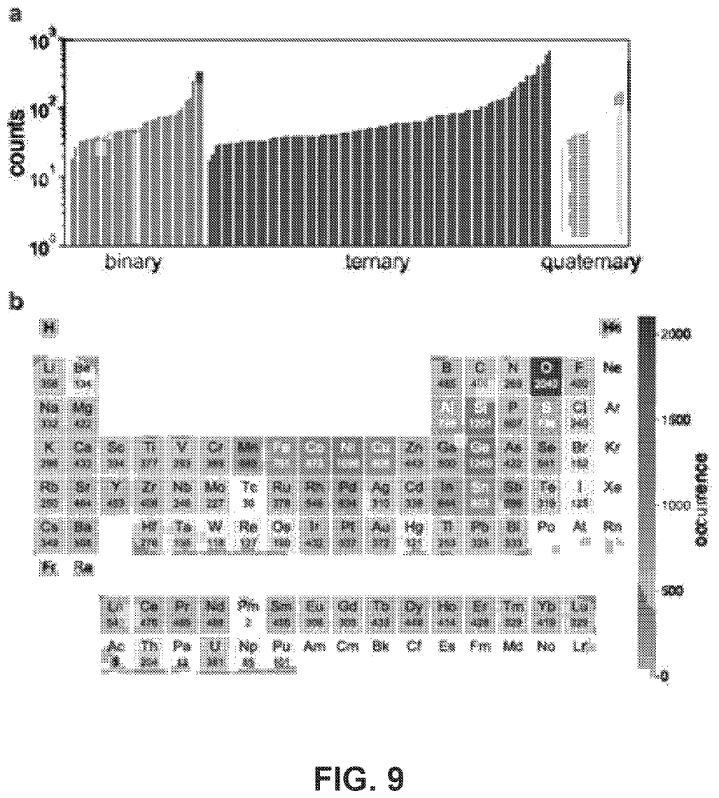

FIG. 9 depicts a statistical distribution of a dataset used for material property prediction, in accordance with some example embodiments. As shown in FIG. 9, the dataset includes a total of 12,277 and 1,672 unique crystals with formation energies and elastic moduli (pre-computed using density functional theory (DFT) ground state calculations), respectively, from the Materials Project. These crystals belong to 144 (35 binary, 91 ternary, and 18 quaternary) common structure prototypes in the Inorganic Crystal Structure Database (ICSD), with each prototype having at least 30 compositions.

FIG. 9(a) shows the distribution of 35 binary, 91 ternary, and 18 quaternary commonly occurring structure prototypes in the dataset. Each bar represents a single structure prototype and, as noted, the minimum of 30 unique compositions for each structure prototype. FIG. 9(b) shows the frequency of each element occurring in the dataset with the elements being color-coded according to the number of occurrences. As shown in FIG. 9(b), oxygen (O) is the most common element. The relatively high frequencies of the transition metal elements such as iron (Fe), cobalt (Co), nickel (Ni), and copper (Cu) can be attributed to the commonly occurring intermetallic structure prototypes. The three most commonly occurring structure prototypes are the ternary intermetallic (ThCr₂Si₂,¹ TiNiSi,² and ZrNiAl³), which have 633, 619, and 484 compositions, respectively.

For each crystal in the dataset shown in FIG. 9 (e.g., rock salt germanium telluride (GeTe)), another crystal with the same prototype but a different composition (e.g., rock salt sodium chloride (NaCl)) may be selected at random and multi-element substitutions (Na→Ge, Cl→Te) may be performed to arrive at an “unrelaxed” initial crystal structure. The Bayesian optimization with symmetry relaxation (BOWSR) algorithm may then applied to obtain a relaxed structure. The unrelaxed, BOWSR-relaxed, and the original DFT-relaxed structures were then provided as inputs for property predictions using MatErials Graph Network (MEGNet) formation energy and elastic moduli models. These MEGNet models may be trained on the DFT-computed formation energies and elastic moduli of 133, 420 and 12,179 crystals, respectively, from the Materials Project.

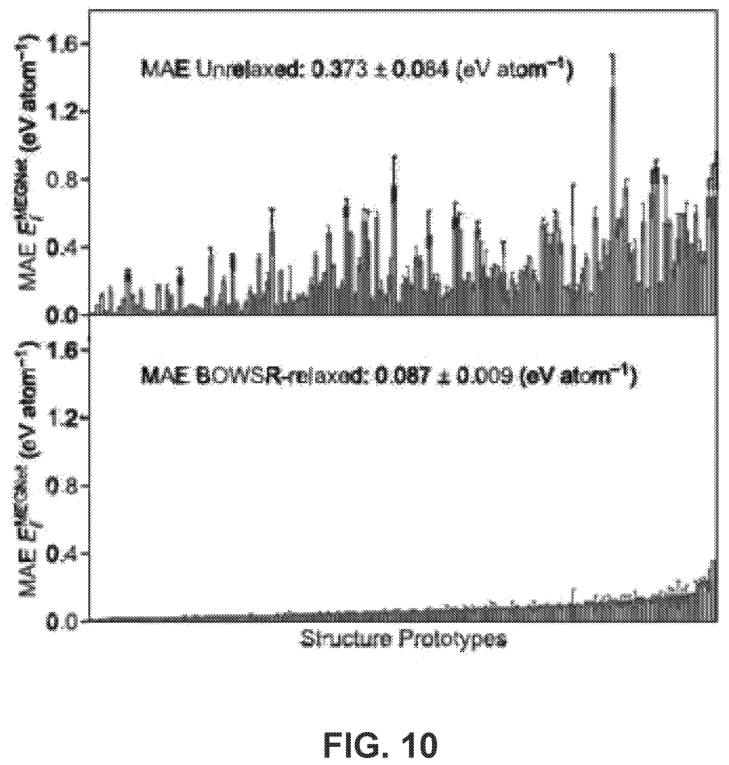

FIG. 10 compares the MEGNet model predictions using the unrelaxed, BOWSR-relaxed, and DFT-relaxed structures as inputs with respect to DFT-computed values. The mean absolute errors (MAEs) of the MEGNet models using the DFT-relaxed structures provide a best-case performance baseline but it should be noted that the MEGNet models were trained using a superset of data from the Materials Project that includes the DFT-relaxed structures. Hence, the reported mean absolute errors of the MEGNet predictions made using DFT-relaxed structures may be much smaller than the previously reported mean absolute errors of these MEGNet models from cross-validation and should not be considered as a metric for MEGNet performance. Using the unrelaxed structures as inputs results in much higher, positively skewed mean absolute errors in the MEGNet formation energy prediction compared to using DFT-relaxed structures. This is because the unrelaxed structures have lattice parameters and atomic positions that can deviate substantially from the ground state DFT-relaxed structures, resulting in higher energies. Using the BOWSR-relaxed structures as inputs reduces the mean absolute errors by a factor of four, from 363 meV atom⁻¹ to 88 meV atom⁻¹. The R2 also substantially increases from 0.67 to 0.96, and the error distribution is Gaussian-like with a mean close to 0. Similarly, large improvements in the MEGNet predictions of the elastic moduli are also observed using the BOWSR-relaxed structures compared to using unrelaxed structures, with MAEs in the log₁₀ K_{VRH} and log₁₀ G_{VRH} reducing by half.

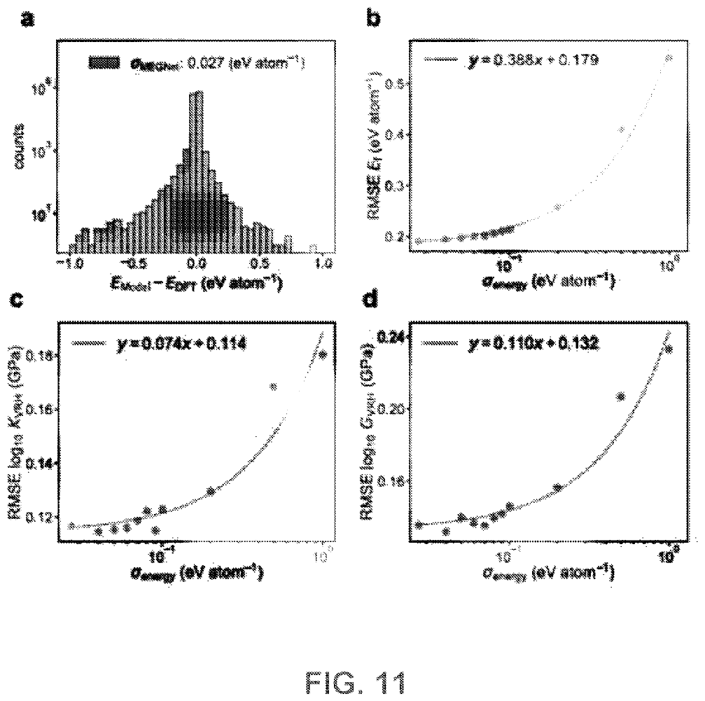

The sensitivity of the Bayesian optimization with symmetry relaxation (BOWSR) algorithm to the initial structures used to perform elemental substitution is tested using four randomly chosen parent structures with different lattice parameters for each prototype. FIG. 11 depicts graphs illustrating the sensitivity of the Bayesian optimization with symmetry relaxation (BOWSR) algorithm to the accuracy of the energy model, in accordance with some example embodiments. For example, FIG. 11(a) shows the error distributions of the MEGNet formation energy model on the training and test data. Both the standard deviation (σ) and root mean square error (RMSE) are 27 meV atom⁻¹. Varying amounts of Gaussian noise are added to the MEGNet formation energy prediction during the BOWSR relaxation process.

The error of the energy model σ_{energy} is then given by \sqrt{σ_{MEGNet}²+σₙₒᵢₛₑ²} where σₙₒᵢₛₑ denotes the standard deviation of the added noise. The root mean square error (RMSE) of the MEGNet-predicted formation energy, bulk modulus, and shear modulus for the BOWSR-relaxed structures are plotted against the error in the energy model and shown in FIGS. 8(b), (c), and (d), respectively. In all cases, linear correlations are observed between the root mean square error (RMSE) of the MEGNet prediction and the error of the energy model, and reasonably low root mean square error (RMSE) in prediction are obtained when σ_{energy} <0.1 eV atom⁻¹. Accordingly, FIG. 11 shows that the BOWSR-relaxed structures exhibit consistently low errors regardless of initial structure selection.

FIG. 11 also shows the sensitivity of the Bayesian optimization with symmetry relaxation (BOWSR) algorithm to the accuracy of the energy evaluator, which is tested by artificially introducing Gaussian noise into the MEGNet formation energy prediction. The energy errors from the BOWSR-relaxed structures are linearly correlated with the errors of the surrogate machine learning model with the root mean square error (RMSE) ranging from 27 to 1000 meV atom⁻¹, which indicates the robustness of the Bayesian optimization (BO) propagation and the broad applicability of the Bayesian optimization with symmetry relaxation (BOWSR) algorithm to any surrogate machine learning models. As shown in FIGS. 11(c) and (d), the same linear correlations are also observed between the elastic moduli errors and the errors of the surrogate machine learning model.

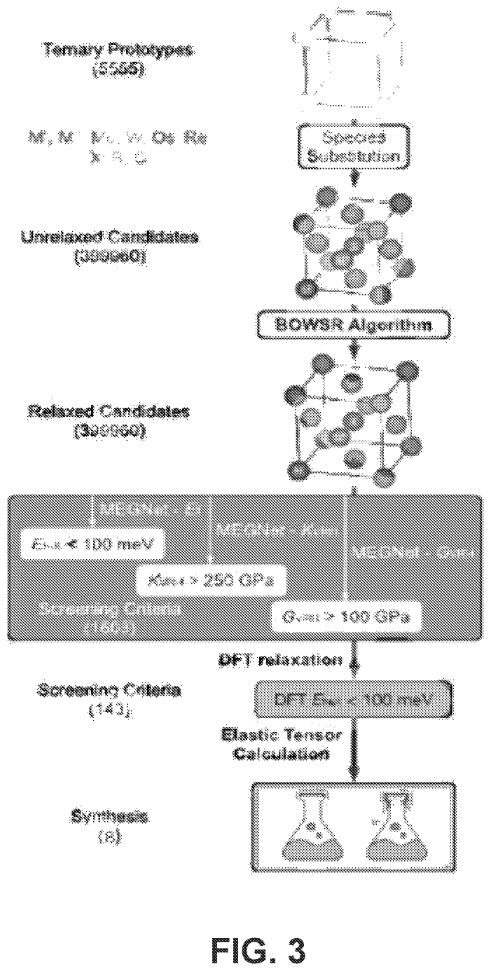

In some example embodiments, the data engine 110, with the structure generator 113 applying the Bayesian optimization with symmetry relaxation (BOWSR) algorithm and the property predictor 115 applying the MatErials Graph Network (MEGNet) models, may screen a large quantity of candidate crystal structures to identify those exhibiting exceptional properties. FIG. 3 depicts a flowchart illustrating an example of a process for material discovery that leverages the Bayesian optimization with symmetry relaxation (BOWSR) algorithm and the MatErials Graph Network (MEGNet) models, in accordance with some example embodiments. As shown in FIG. 3, the search may be focused on 12 ternary M′ₓM″_{y}X_{z} chemical spaces, where M′, M′=molybdenum (Mo), tungsten (W), osmium (Os), or rhenium (Re) and X=boron (B) or carbon (C). These elements were selected based on their common occurrences in ultra-incompressible hard binary compounds. By combinatorially applying elemental substitutions to 5,555 ternary structures prototypes in the Inorganic Crystal Structure Database (ICSD), 399,960 candidates were generated and relaxed using the BOWSR algorithm with the MEGNet energy model. The BOWSR-relaxed candidates were then screened for stability and mechanical properties using MEGNet models (e.g., for formation energy and elastic moduli). The stability metric used was the energy above hull Eₕᵤₗₗ^{MEGNet}, which was computed using the predicted formation energy E_{f}^{MEGNet} with the 0 K phase diagram in the Materials Project database. At this intermediate stage, a relatively generous threshold of Eₕᵤₗₗ^{MEGNet} <100 meV atom⁻¹ was used to obtain candidates that are likely to be hull thermodynamic stable. Of these, candidates with relatively high MEGNet-predicted bulk and shear moduli (K_{VRH}^{WENet}>250 GPa and Gₕᵤₗₗ^{MEGNet} >100 GPa) were identified. Similar to the stability criterion, the mechanical criteria used are slightly lower than the conventional threshold for ultra-incompressibility to account for the higher mean absolute error (MAE) of the MEGNet elastic moduli predictions. Density functional theory (DFT) based relaxations and energy calculations were then carried out on the 1,603 candidates that passed all three machine learning based screening criteria. Subsequently, expensive density functional theory (DFT) based elastic tensor calculations were performed on the 143 candidates that have Eₕᵤₗₗ^{DFT}<100 meV atom⁻¹.

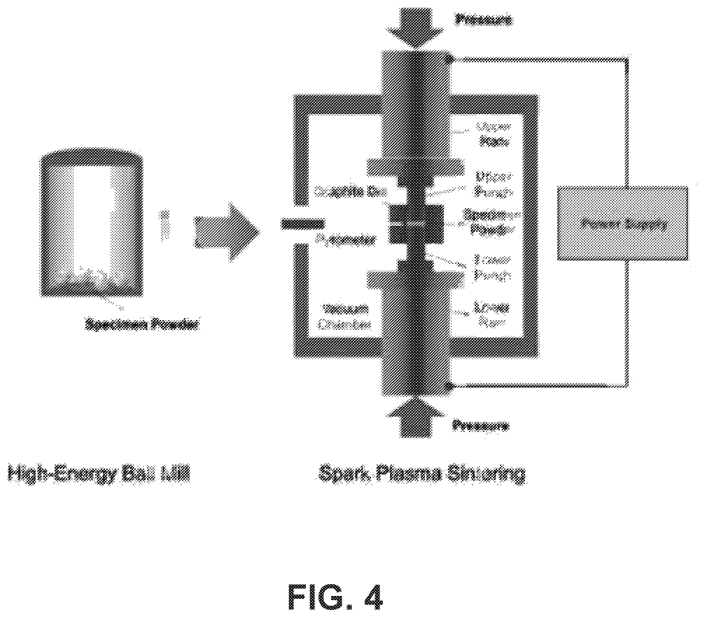

Table 1 below summarizes the computed elastic properties of the top ten candidates with the highest computed bulk modulus together with other well-known ultra-incompressible materials. Attempts were then made to synthesize all ten candidates with eight unique compositions via in-situ reactive spark plasma sintering (SPS) using elemental precursors in the appropriate ratios. FIG. 4 depicts a schematic diagram illustrating an example of a specimen preparation process in which specimen powders were first high-energy ball milled and then densified into pellets via spark plasma sintering.

TABLE 1
DFT-computed bulk modulus (K_{VRH}), shear modulus (G_{VRH}),
Young's modulus (E_{VRH}), Poisson's ratio (ν) and energy above
hull (Eₕᵤₗₗ) for the top 10 candidates with regard to K_{VRH} in descending
order. MoWC₂ and ReWB are bolded as they are successfully
synthesized by experiments. Some of the known ultra-incompressible
materials are used as references.

| | | | | | Eₕᵤₗₗ |
|---|---|---|---|---|---|
| | K_{VRH} | G_{VRH} | E_{VRH} | | (meV |
| | (GPa) | (GPa) | (GPa) | ν | atom¹) |
| Candidates | | | | | |
| ReOsB (P6m2) | 370.7 | 241.3 | 594.7 | 0.233 | 31.7 |
| ReOsB₂ (P6₃/mmc) | 367.3 | 220.9 | 552.0 | 0.250 | 87.8 |
| MoWC₂ (P6₃/mmc) | 357.9 | 260.5 | 628.8 | 0.207 | 96.3 |
| ReWB (Fddd) | 356.8 | 176.9 | 455.5 | 0.287 | 20.6 |
| Re₁₃WB₉ (P6m2) | 353.1 | 177.0 | 455.1 | 0.285 | 88.4 |
| ReWB (Pca2₁) | 352.6 | 144.1 | 380.4 | 0.320 | 33.1 |
| OsWB (Pbam) | 351.1 | 183.1 | 467.9 | 0.278 | 43.3 |
| ReWB (Cmcc) | 350.9 | 161.5 | 420.1 | 0.301 | 32.6 |
| Re₈W₇B₈ (P6/m) | 348.4 | 182.8 | 466.8 | 0.277 | 22.2 |
| ReW₂B₂ (P4/mbm) | 345.8 | 156.0 | 406.8 | 0.304 | 72.1 |
| Known incompressible | | | | | |
| materials | | | | | |
| C (Fd3m) | 430.3 | 503.6 | 1086.9 | 0.079 | 136.4 |
| WC (P6m2) | 380.8 | 280.0 | 677.8 | 0.210 | 1.1 |
| BN (F43m) | 370.1 | 382.8 | 852.4 | 0.116 | 77.3 |
| ReB₂ (P6₃/mmc) | 334.9 | 272.3 | 642.7 | 0.180 | 4.7 |

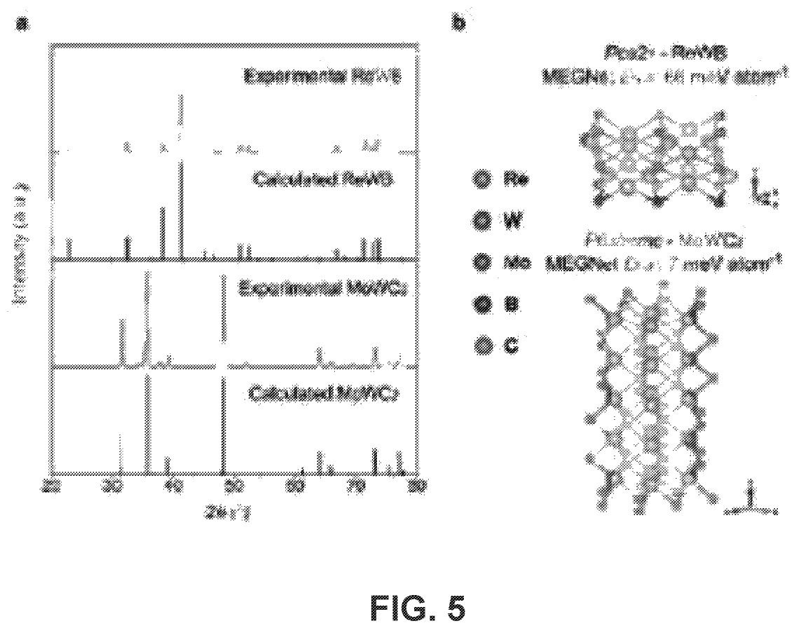

Two crystals from Table 1, MoWC₂ (P6₃/mmc) and ReWB (Pca2₁), were successfully synthesized and confirmed via X-ray power diffraction as single phase. Henceforth, the two novel phases of MoWC₂ (P6₃/mmc) and ReWB (Pca2₁) will be referred to as simply as MoWC₂ and ReWB, respectively. FIG. 5(a) shows the measured and calculated X-ray power diffraction (XRD) patterns of MoWC₂ and ReWB. The crystal structures and space group of these two materials are shown in FIG. 5(b). The predicted energy above hull for ReWB and MoWC₂ are 66 and 7 meV atom⁻¹, respectively.

Table 2 below shows a comparison of the BOWSR-relaxed, DFT-relaxed, and experimentally measured lattice parameters for MoWC₂ (P6₃/mmc) and ReWB (Pca2₁). The lattice angles are constrained to fixed values by virtue of their orthorhombic and hexagonal crystal systems.

TABLE 2

| | | BOWSR-relaxed | DFT-relaxed | Experimental |
|---|---|---|---|---|
| ReWB (Pca2₁) | a (A) | 5.182 | 5.534 | 5.519 |
| | b (A) | 4.864 | 5.537 | 5.506 |
| | c (A) | 5.532 | 4.729 | 1.729 |
| MoWC₂ | a (A) | 2.891 | 2.928 | 2.907 |
| (P6₃/mmc) | c (A) | 11.580 | 11.448 | 11.372 |

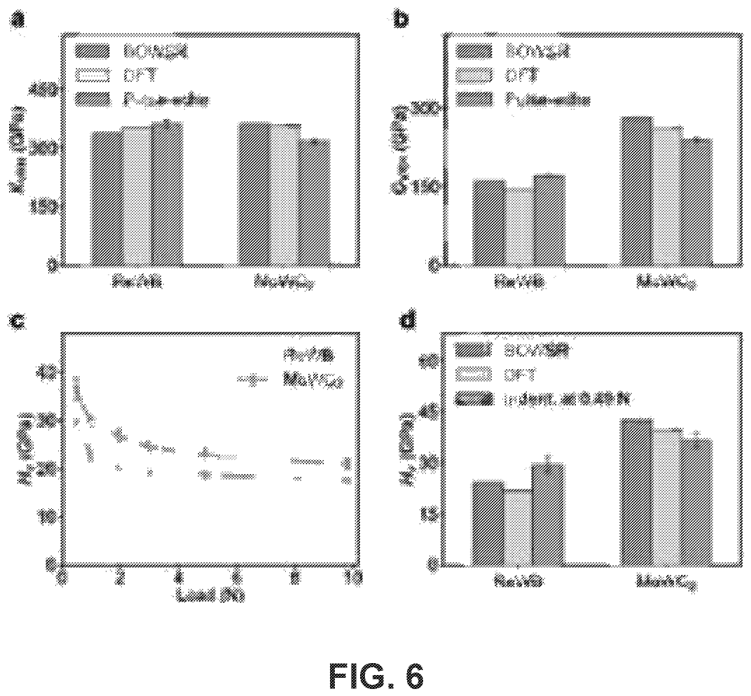

The mechanical properties of MoWC₂ and ReWB were measured using the pulse-echo method. As shown in FIGS. 6(a) and (b), the experimentally-measured bulk and shear moduli are consistent with MEGNet and density functional theory (DFT) based predictions. Both new materials exhibit ultra-incompressibility, with bulk modulus close to or larger than 300 GPa.³⁰ MoWC₂ also exhibits high estimated Vickers hardnesses Hᵥ of 36.6 at 0.49 N indentation load and 20.9 GPa at 9.8 N load (see FIG. 6(c)). ReWB has a comparatively lower measured hardness of 29.5 at 0.49 N load and 17.6 GPa at 9.8 N load. The Hᵥ values at 0.49 N load are within 20-25% of those derived from the MEGNet and DFT predicted shear moduli via the empirical relation Hᵥ=0.151G,³⁶ as shown in FIG. 6(d).

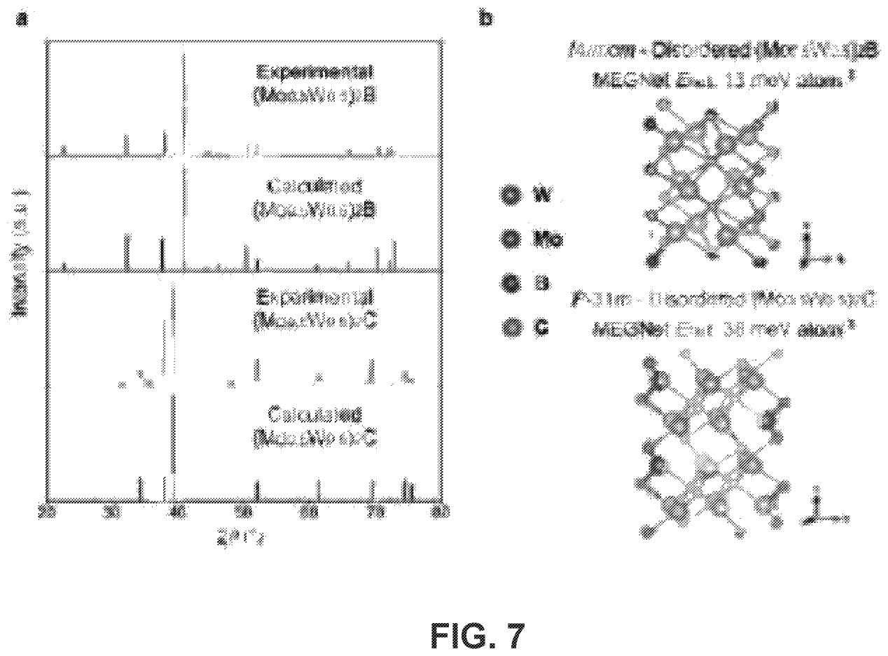

FIG. 7(a) depicts a graph illustrating the measured and calculated X-ray power diffraction (XRD) patterns of (Mo_{0.5}B_{0.5})₂B and (Mo_{0.5}W_{0.5})₂C. Crystal structures and space group of these two materials are shown in FIG. 7(b). The predicted energy above hull for (Mo_{0.5}B_{0.5})₂B and (Mo_{0.5}W_{0.5})₂C are 13 and 38 meV atom⁻¹, respectively.

Table 3 below provides a detailed description of the ordered Pca2₁-ReWB crystal structure.

TABLE 3

| | | Elemental | | | |
|---|---|---|---|---|---|
| Formula | Lattice | Occupancies | Atomic Positions | | |
| ReWB | a: 5.518 Å | Re | 0.9229 | 0.9174 | 0.6184 |
| | b: 5.506 Å | Re | 0.0771 | 0.0826 | 0.1184 |
| | c: 4.701 Å | Re | 0.5771 | 0.9174 | 0.1184 |
| | α: 90° | Re | 0.4229 | 0.0826 | 0.6184 |
| | β: 90° | W | 0.4155 | 0.4185 | 0.1110 |
| | γ: 90° | W | 0.5845 | 0.5815 | 0.6110 |
| | | W | 0.0845 | 0.4185 | 0.6110 |
| | | W | 0.9155 | 0.5815 | 0.1110 |
| | | B | 0.2481 | 0.7564 | 0.3643 |
| | | B | 0.7519 | 0.2436 | 0.8643 |
| | | B | 0.2519 | 0.7564 | 0.8643 |
| | | B | 0.7481 | 0.2436 | 0.3643 |

Table 4 below provides a detailed description of the P6₃/mmc-MoWC₂ crystal structure.

TABLE 4

| | | Elemental | | | |
|---|---|---|---|---|---|
| Formula | Lattice | Occupancies | Atomic Positions | | |
| MoWC₂ | a: 2.907 Å | Mo | 0.0000 | 0.0000 | 0.5000 |
| | b: 2.907 Å | Mo | 0.0000 | 0.0000 | 0.0000 |
| | c: 11.372 Å | W | 0.0000 | 0.0000 | 0.2500 |
| | α: 90° | W | 0.0000 | 0.0000 | 0.7500 |
| | β: 90° | C | 0.3333 | 0.6667 | 0.3730 |
| | γ: 120° | C | 0.6667 | 0.3333 | 0.6270 |
| | | C | 0.6667 | 0.3333 | 0.8730 |
| | | C | 0.3333 | 0.6667 | 0.1270 |

Table 5 below provides a detailed description of the I4/mcm-(Mo_{0.5}W_{0.5})B crystal structure.

TABLE 5

| | | Elemental | | | |
|---|---|---|---|---|---|
| Formula | Lattice | Occupancies | Atomic Positions | | |
| (Mo_{0.5}W_{0.5})B | a: 5.551 Å | 0.5 Mo:0.5 W | 0.6777 | 0.1777 | 0.0000 |
| | b: 5.551 Å | 0.5 Mo:0.5 W | 0.3223 | 0.8223 | 0.0000 |
| | c: 4.775 Å | 0.5 Mo:0.5 W | 0.8223 | 0.6777 | 0.0000 |
| | α: 90° | 0.5 Mo:0.5 W | 0.1777 | 0.3223 | 0.0000 |
| | β: 90° | 0.5 Mo:0.5 W | 0.6777 | 0.8223 | 0.5000 |
| | γ: 90° | 0.5 Mo:0.5 W | 0.3223 | 0.1777 | 0.5000 |
| | | 0.5 Mo:0.5 W | 0.8223 | 0.3223 | 0.5000 |
| | | 0.5 Mo:0.5 W | 0.1777 | 0.6777 | 0.5000 |
| | | B | 0.0000 | 0.0000 | 0.7500 |
| | | B | 0.0000 | 0.0000 | 0.2500 |
| | | B | 0.5000 | 0.5000 | 0.2500 |
| | | B | 0.5000 | 0.5000 | 0.7500 |

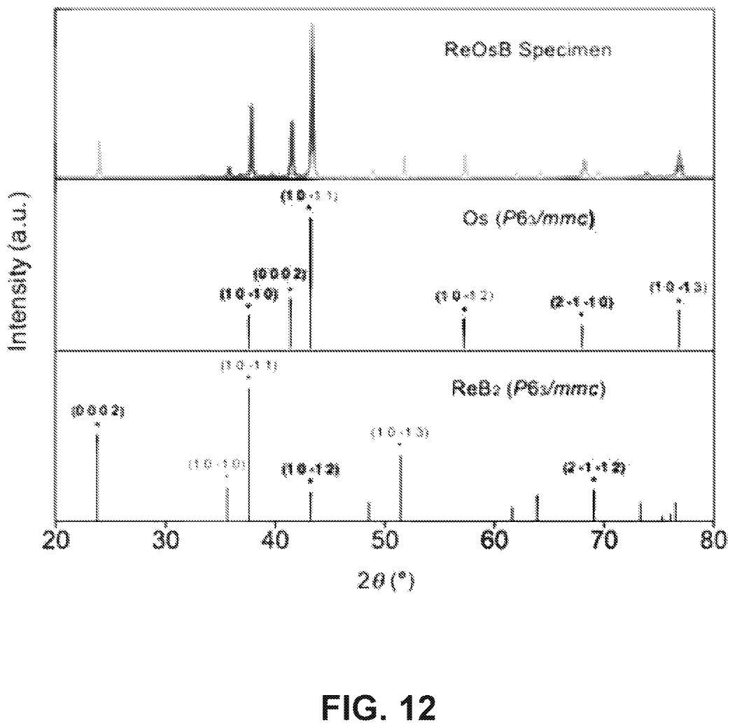

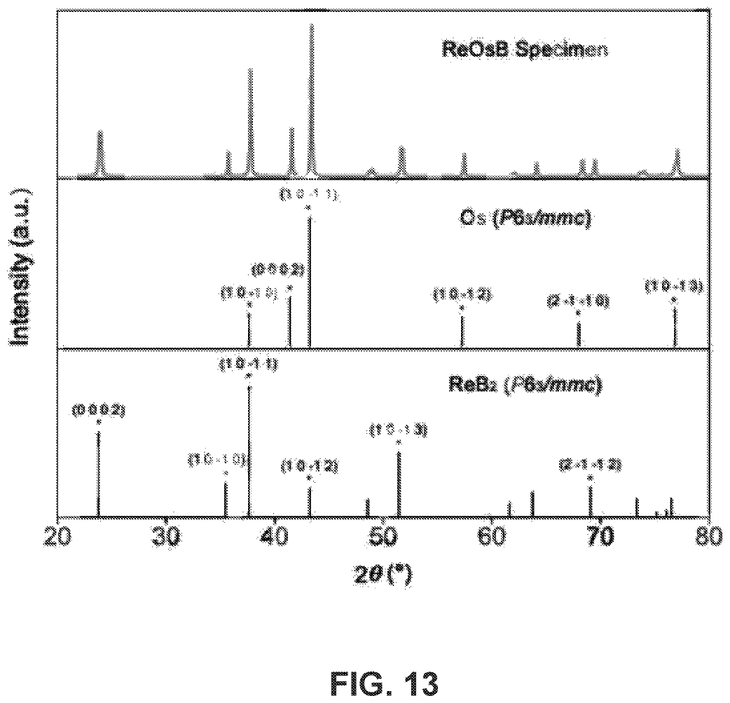

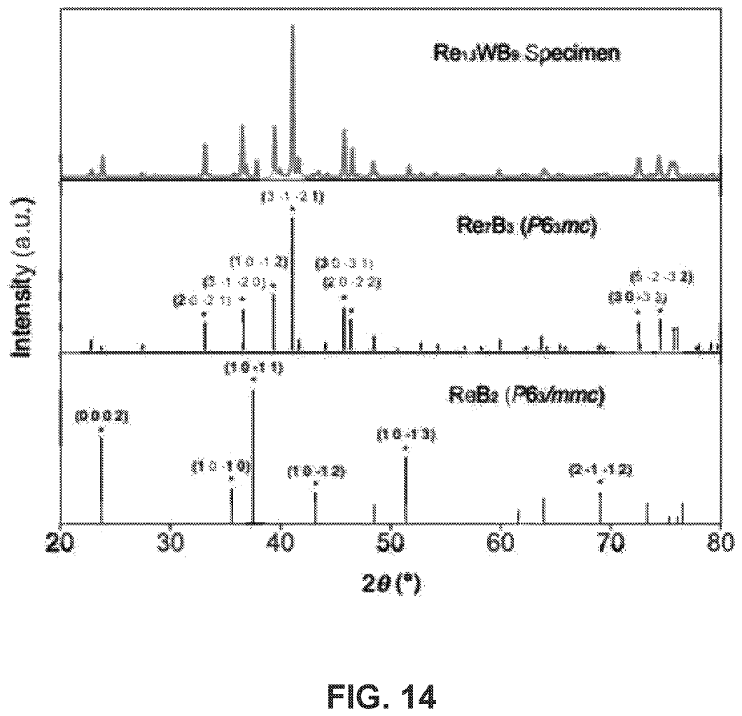

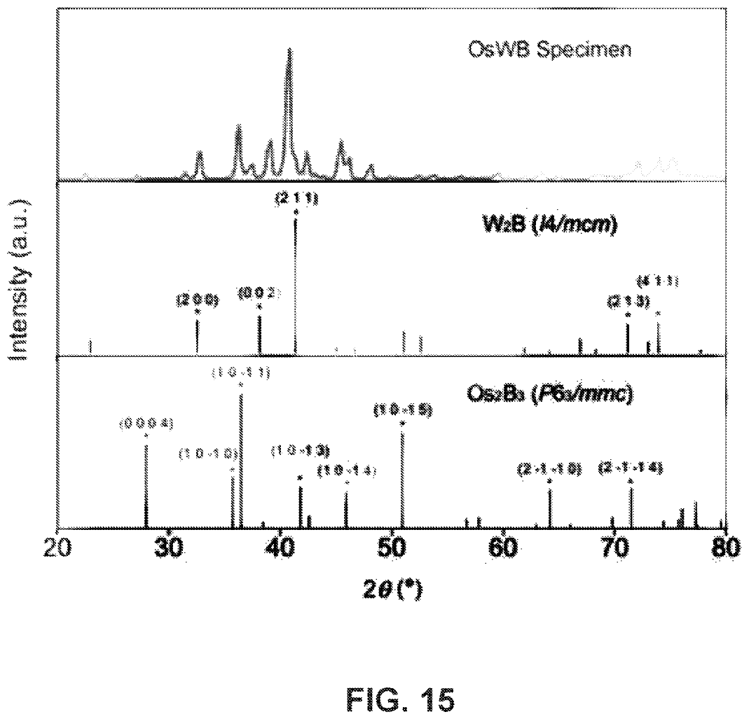

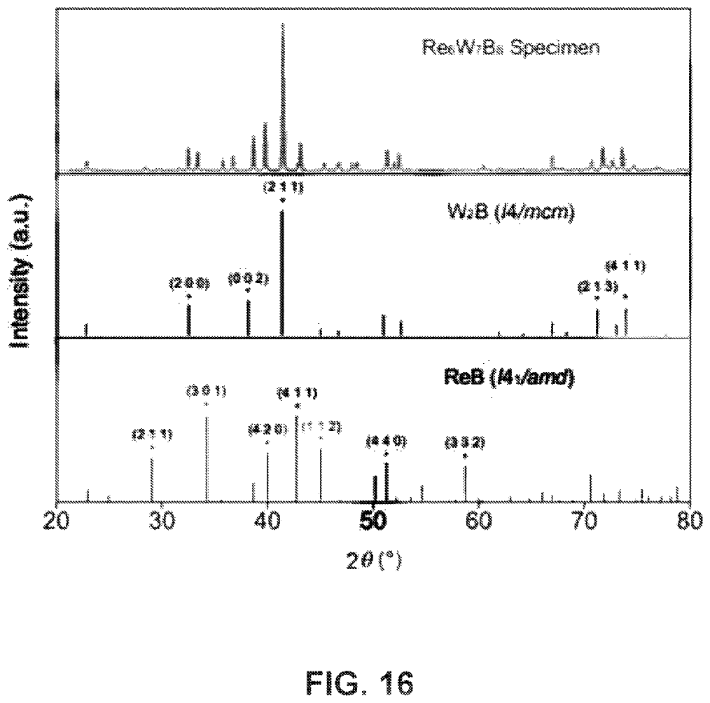

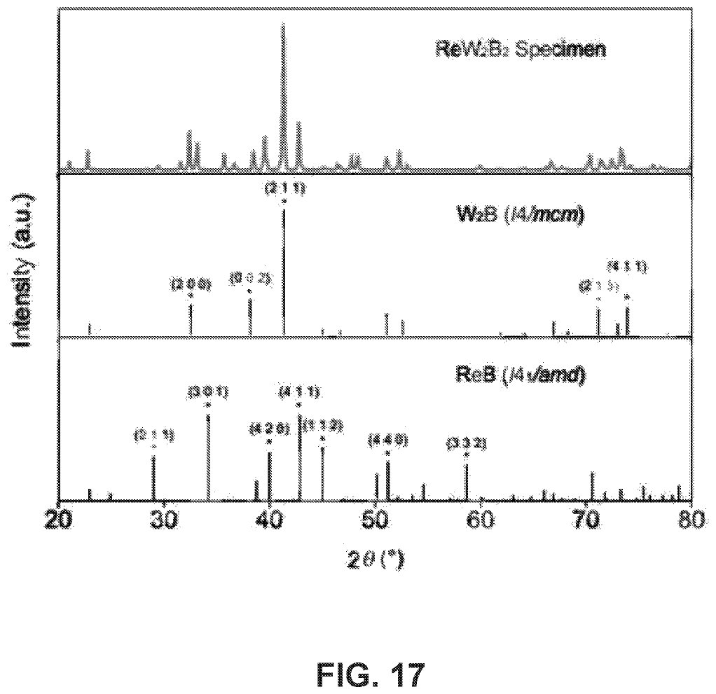

Meanwhile the synthesis of the other six compositions yielded multiple phases. For example, FIG. 12 depicts a graph illustrating the measured X-ray power diffraction (XRD) patterns of the ReOsB specimen, which exhibits two major phases Os (P6₃/mmc) and ReB₂ (P6₃/mmc). FIG. 13 depicts a graph illustrating the measured X-ray power diffraction (XRD) patterns of the ReOsB₂ specimen, which exhibits two major phases: Os (P6₃/mmc) and ReB₂ (P6₃/mmc). FIG. 14 depicts a graph illustrating the measured X-ray power diffraction (XRD) patterns of the Re₁₃WB₉ specimen, which exhibits a primary phase of Re₇B₃ (P6₃mc) and a secondary phase of ReB₂ (P6₃/mmc). FIG. 15 depicts a graph illustrating the measured X-ray power diffraction (XRD) patterns of the OSWB specimen, which exhibits multiple phases including the W₂B (14/mcm) and Os₂B₃ (P6₃/mmc). FIG. 16 depicts a graph illustrating the measured X-ray power diffraction (XRD) patterns of the Re₆ W₇B₈ specimen, which exhibits a primary phase of W₂B (14/mcm) and a secondary phase of ReB (I4₁/amd). FIG. 17 depicts graphs illustrating the measured X-ray power diffraction (XRD) patterns of the ReW₂B₂ specimen, which exhibits a primary phase of W₂B (I4/mcm) and a secondary phase of ReB (I4₁/amd). It should be appreciated that the major peaks in FIGS. 12-17 are indexed for reference.

Many material properties, such as formation energies and mechanical properties, exhibit a strong dependence on the crystal structure. However, as noted, obtaining equilibrium crystal structures as inputs that enables a machine learning based model to perform accurate property prediction still requires expensive ab initio computations. By coupling an accurate graph based deep learning energy model, such as the MatErials Graph Network (MEGNet) formation energy model, with Bayesian optimization of symmetry-constrained parameters, the resulting algorithm can reasonably approximate equilibrium structures with substantial improvements in machine learning enabled property predictions. For example, a combination of the Bayesian optimization with symmetry relaxation (BOWSR) algorithm and the MatErials Graph Network (MEGNet) models enables the rapid screening of approximately 400,000 candidate crystal structures for stability and exceptional mechanical properties, a solution space that is 103 to 104 orders of magnitude larger than that accessible by high-throughput density functional theory (DFT) based calculations.

Referring again to FIG. 1, the design engine 110 may include the structure generator 113, which generates, for each input crystal structure, a corresponding equilibrium crystal structure by applying the Bayesian optimization with symmetry relaxation (BOWSR) algorithm in order to “relax” each input crystal structure along its potential energy surfaces. Geometry relaxation of a crystal structure may require the optimization of up to (3N+6) quantity of variables corresponding to six lattice parameters and three fractional coordinates for each of the N quantity of atoms. By constraining the symmetry to remain unchanged during relaxation can reduce the number of independent variables considerably. A variety of search algorithms, such as the open-source Spglib library, may be used for symmetry determination. The search for optimized symmetry-constrained lattice parameters and atomic coordinates that minimize the total energy may then be carried out via Bayesian optimization (BO). The changes in the variables were used as the optimization inputs to reduce the tendency of the Bayesian optimization process being dominated by parameters with large magnitudes.

Using a Latin hypercube sampling, a set of training observations D˜{(xᵢ, U(xᵢ)) i=1: m} were initialized, wherein xᵢ denotes the m independent lattice parameters and atomic coordinates while U(.) denotes the energy of the corresponding structure evaluated by the surrogate machine learning model (see Equations (1) and (2) above). The Bayesian optimization (BO) strategy may include two operations.

First, a Gaussian process (GP) model may be trained on the initialized training observations D to approximate the energy U(x). The Rational Quadratic kernel may be adopted as the covariance function of the Gaussian process (GP) model. The noise level of Gaussian process (GP) model may be set to the root mean square error (RMSE) of the energy model.

Second, the acquisition function that balances the exploitation and exploration of the search space is calculated for samples in the search space apart from the training observations. The candidate crystal structure with an optimal acquisition function is proposed for subsequent evaluation, for example, by the property predictor 115 applying the MatErials Graph Network (MEGNet) formation energy model. In this context, exploitation represents the samples with high predicted mean from the Gaussian process (GP) model whereas exploration accounts for the samples with high predictive uncertainty. Here, the acquisition function may correspond to the expected improvement, which can be analytically expressed as Equations (4)-(5) below.

$$E [ I ( x ) ] = ( μ ( x ) - U ( {x}^{+} ) - ξ ) \cdot Φ ( Z ) + σ ( x ) \cdot ϕ ( Z ) \qquad ( 3 )$$

$$and {x}^{+} = {arg min}_{i = 1 , … , n} U ( {x}_{i} ) \qquad ( 4 )$$

$$Z = \frac{μ ( x ) - U ( {x}^{+} ) - ξ}{σ ( x )} \qquad ( 5 )$$

wherein μ(x) denotes the mean deviation of the posterior distribution on x from the Gaussian process (GP) model, σ(x) denotes the standard deviation of the posterior distribution on x from the Gaussian process (GP) model, Φ(x) is the cumulative distribution function (CDF), and ϕ(x) is the probability density function (PDF). The parameter ξ can be tuned to balance the trade-off between the first term (exploitation) and the second term (exploration) in Equation (3). Until the maximum number of iteration steps is reached, the sample with optimal acquisition function may be iteratively augmented to the training observations and used to update the Gaussian process (GP) surrogate model in the next loop.

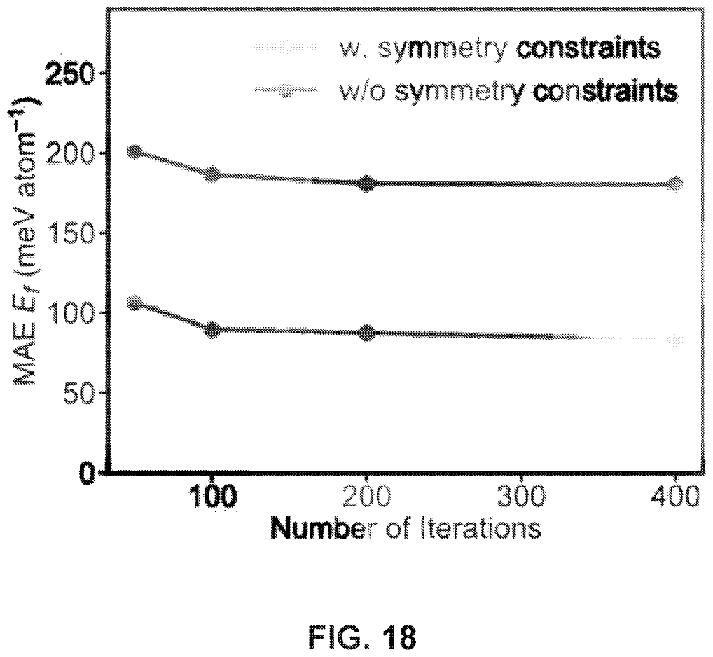

It should be noted that by removing the symmetry constraint, i.e., treating all crystals as having triclinic P1 symmetry, the Bayesian optimization (BO) yields much higher errors than that with symmetry constraints. This phenomenon, which is graphically illustrated in FIG. 18, can be attributed to the limitation of Bayesian optimization (BO) in optimizing the high dimensional parameter space that scales linearly with the number of atoms in crystals without symmetry. As such, in practice, the number of initialized training observations and the maximum number of iterations may be set to a threshold (e.g., 100) to achieve the best trade-off between accuracy and efficiency (see FIG. 11).

For property predictions, the parameter ξ may be set to favor exploitation (e.g., 0) as the dataset has been well explored by density functional theory (DFT) based calculations. For the discovery of new incompressible materials, the parameter ξ may be set to encourage at least some exploration (e.g., 0.1), thus allowing the Bayesian optimization (BO) to have a higher likelihood of finding the global energy minimum in the exploration space with higher predictive uncertainty. In addition, the search space for optimized changes in the fractional coordinates, lattice lengths, and lattice angles variables may be set to 0.2, 1.2 Å, and 5°, respectively. The resulting search space may be sufficiently large to have a reasonably high likelihood of including the global energy minimum while not overly large as to be computationally intractable.

Referring again to FIG. 1, the design engine 110 may include the property predictor 115, which determines, for each equilibrium crystal structure generated by the structure generator 113, one or more material properties such as formation energies, elastic moduli, and/or the like. As noted, in some cases, the property predictor 115 may apply a machine learning based property prediction model such as a graph based deep learning energy model in which atoms and bonds in crystal structures are represented as nodes and edges in a mathematical graph. The aforementioned MatErials Graph Network (MEGNet) models, for example, may include three graph convolutional layers with neurons to perform each update function, and a shifted softplus function for the non-linear activation function. A set2set readout function with two passes may be used after the graph convolution operations. The cutoff radius for constructing the neighbor graph was 5 Å. The MEGNet formation energy (E_{f}) and elasticity (K_{VRH} and G_{VRH}) models were trained using the 2019.4.1 version of Materials Project database containing 133,420 structure-formation energy and 12,179 structure-bulk/shear modulus data pairs. Each dataset may be split into training data, validation data, and testing data at a ratio of 80%:10%:10%. A batch size of 128 structures were using during the training of the model, with the initial learning rate set to 0.001 in the Adam optimizer. The models were trained for a maximum of 1500 epochs with an early stopping callback, which terminates the model training if the validation error does not reduce for 500 consecutive steps. The mean absolute errors (MAEs) of E_{f}, log₁₀ (K_{VRH}) and log 10 (G_{VRH}) models in test data are 26 meV atom⁻¹, 0.07, and 0.12, respectively.

The density functional theory (DFT) relaxations, energy calculations, and elastic tensor calculations for a small number of candidates that passed the machine learning based screening were carried out using Vienna ab initio simulation package (VASP) within the projector augmented wave approach. The exchange-correlation interaction was described using the Perdew-Burke-Ernzerhof (PBE) generalized gradient approximation (GGA) functional for structural relaxations and energy calculations. The plane wave energy cutoff was set to 520 eV, and the k-point density of at least 1,000 per number of atoms was used. All structures were relaxed with energies and forces converged to 10-5 eV and 0.01 eV/A, respectively, consistent with the calculation setting used in the Materials Project.¹ The elastic tensor calculations were performed using existing procedures. A tighter energy convergence criterion of 10⁻⁷ eV was used, and strains with magnitude of (−1%, −0.5%, 0.5%, 1%) were applied to each of the 6 independent components of strain tensor.

In accordance with the specimen preparation process shown in FIG. 4, bulk specimens of candidate crystal structures ReOsB, ReOsB₂, MoWC₂, ReWB, Re₁₃WB₉, OsWB, Re₆ W₇B₈, and ReW₂B₂ may be synthesized via in-situ reactive spark plasma sintering (SPS). Elemental powders of Mo, W (>99.5% purity, ˜325 mesh, Alfa Aesar), Re (˜99.99% purity, ˜325 mesh, Strem Chemicals), Os (˜99.8% purity, ˜200 mesh, Alfa Aesar), boron (˜99% purity, 1-2 μm, US Research Nanomaterials), and graphite (˜99.9% purity, 0.4-1.2 μm, US Research Nanomaterials) were utilized as precursors. For each composition, stoichiometric amounts of elemental powders were weighted out in batches of 5 grams. The powders were first mixed by a vortex mixer, and then high energy ball milled (HEBM) in a Spex 8000D mill (SpexCertPrep) by tungsten carbide lined stainless steel jars as well as 11.2 mm tungsten carbide milling media (ball-to-powder ratio≈4.5:1) for 50 minutes. 0.05 grams or ˜1 wt % of stearic acid was used as lubricant in the milling process. After HEMB, the as-milled powder mix-tures were loaded into 10 mm graphite dies lined with graphite foils in batches of 2.5 grams, and subsequently consolidated into dense pellets via spark plasma sintering (SPS) in vacuum (<10⁻² Torr) by a Thermal Technologies 3000 series spark plasma sintering (SPS) machine. The HEBM and powder handing were conducted in an argon atmosphere (with O₂ level <10 ppm) to prevent oxidation.

During the spark plasma sintering (SPS) process, specimens were initially heated to 1400° C. at a rate of 100° C./min under constant pressure of 10 MPa. For the final densification, the temperature was subsequently raised at a constant rate of 30° C./min to a final isothermal sintering temperature, which was set at different levels for different target compositions—1800° C. (ReWB), 1700° C. (MoWC₂ and Re₆W₇B₈), 1600° C. (Re₁₃WB₉ and ReW₂B₂), or 1500° ° C. (ReOsB, ReOsB₂, and OsWB), and maintained isothermally for 10 min. Meanwhile, the pressure was increased to 50 MPa at a ramp rate of 5 MPa/min. The final densification temperature was optimized for each specimen to achieve a high relative density while prevent specimen melting due to overheating. The in-situ reactions between elemental precursors likely took place during the initial temperature ramping. After sintering, the specimens were cooled down naturally inside the spark plasma sintering (SPS) machine (with power off).

Sintered specimens were first ground to remove the carbon-contaminated surface layer from the graphite tooling, and polished for further characterizations. X-ray power diffraction (XRD) experiments were conducted using a Rigaku Miniflex diffractometer with the Cu Kα radiation at 30 kV and 15 mA. The Vickers microhardness tests were carried out on a LECO diamond microindentor with loading force varying from 0.49 N (50 gf) to 9.8 N (1 kgf) and constant holding time of 15 s, abiding by the ASTM Standard C1327. Over 20 measurements at different locations were conducted for each specimen at each indentation load to ensure statistical validity and minimize the microstructural and grain boundary effects. In particular, over 30 measurements were conducted for each specimen at 9.8 N indentation load.

The Young's and shear moduli of the specimens were calculated from the ultrasonic velocities measured with a Tektronix TDS 420A digital oscilloscope, following the ASTM standard A494-15. Multiple measurements were conducted at different locations.

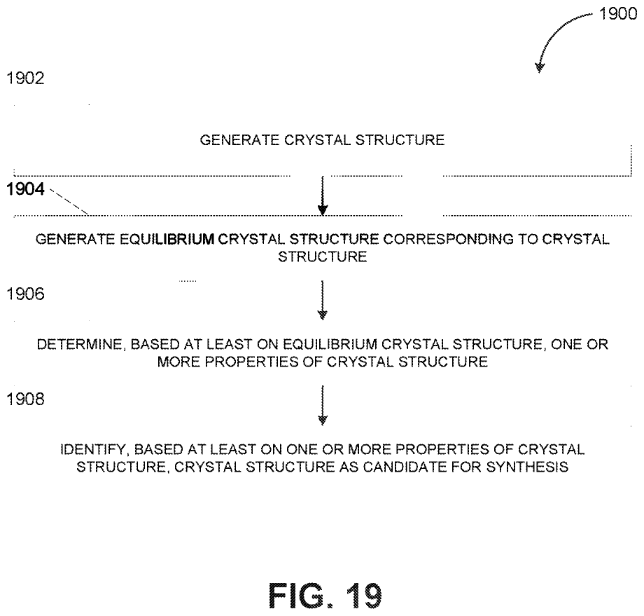

FIG. 19 depicts a flowchart illustrating an example of a process 1900 for machine learning enabled material property prediction, in accordance with some example embodiments. Referring to FIGS. 1 and 19, the process 1900 may be performed by the design engine 110.

At 1902, the design engine 110 may generate a crystal structure. For example, the design engine 110, for example, the structure generator 113, may generate a crystal structure by performing an elemental substitution. As noted, elemental substitution may be a chemically intuitive approach to deriving potential new compounds. For example, the rock salt lithium chloride (LiCl) can be derived from the rock salt sodium chloride (NaCl) by substituting sodium (Na) for the chemically similar element lithium (Li). Accordingly, the structure generator 113 may perform elemental substitution to generate, for example, binary crystal structures, ternary crystal structures, and quaternary crystal structures.

At 1904, the design engine 110 may generate an equilibrium crystal structure corresponding to the crystal structure. In some example embodiments, the design engine 110, for example, the structure generator 113, may apply a first machine learning model trained generate an equilibrium crystal structure having a minimal total energy. For example, the structure generator 113 may generate the equilibrium crystal structure by at least applying the Bayesian optimization with symmetry relaxation (BOWSR) algorithm to “relax” the crystal structure along its potential energy surfaces. As noted, with the Bayesian optimization with symmetry relaxation (BOWSR) algorithm, the symmetry (space group) of the lattice and the Wyckoff positions of the atoms may be constrained during the relaxation process. For instance, the equilibrium crystal structure may be determined by varying the independent lattice parameters and atomic coordinates of the crystal structure while minimizing the overall energy of the crystal structure.

In some example embodiments, each iteration of Bayesian optimization (e.g., for up to a threshold quantity of iterations such as 100) may generate a variation of the initial crystal structure that is constrained with respect to the symmetry (space group) of the lattice and the Wyckoff positions of the constituent atoms. Another algorithm, such as a MatErials Graph Network (MEGNet) formation energy model, may then be applied to determine the formation energy of the variation of the initial crystal structure. Each successive iteration of the Bayesian optimization may include exploring or exploiting the solution space of possible crystal structures for a variation of the initial crystal structure with a lower formation energy.

At 1906, the design engine 110 may determine, based at least on the equilibrium crystal structure, one or more properties of the crystal structure. In some example embodiments, the design engine 110, for example, the property predictor 115, may apply a second machine learning model trained determine one or more properties of the equilibrium crystal structure generated by the structure generator 113. For example, the property predictor 115 may apply the second machine learning model to determine one or more mechanical properties, chemical properties, electrical properties, thermal properties, optical properties, and magnetic properties of the equilibrium crystal structure generated by the structure generator 113. In one example, the property predictor 115 may apply the MatErials Graph Network (MEGNet) models for formation energy and elastic moduli to identify crystal structures exhibiting exceptional mechanical properties such as ultra-incompressibility.

At 1908, the design engine 110 may identify, based at least on the one or more properties of the crystal structure, the crystal structure as a candidate for synthesis. In some example embodiments, crystal structures determined to exhibit certain exceptional (or other desirable) properties, such as ultra-incompressibility, may be identified as candidates for synthesis. For example, the candidate crystal structures and their corresponding properties may be displayed as a part of the user interface 125 at the client device 120. Moreover, in some cases, at least some of the candidate crystal structures shown in Table 1 as exhibiting ultra-incompressibility may undergo synthesis, for example, via in-situ reactive spark plasma sintering.

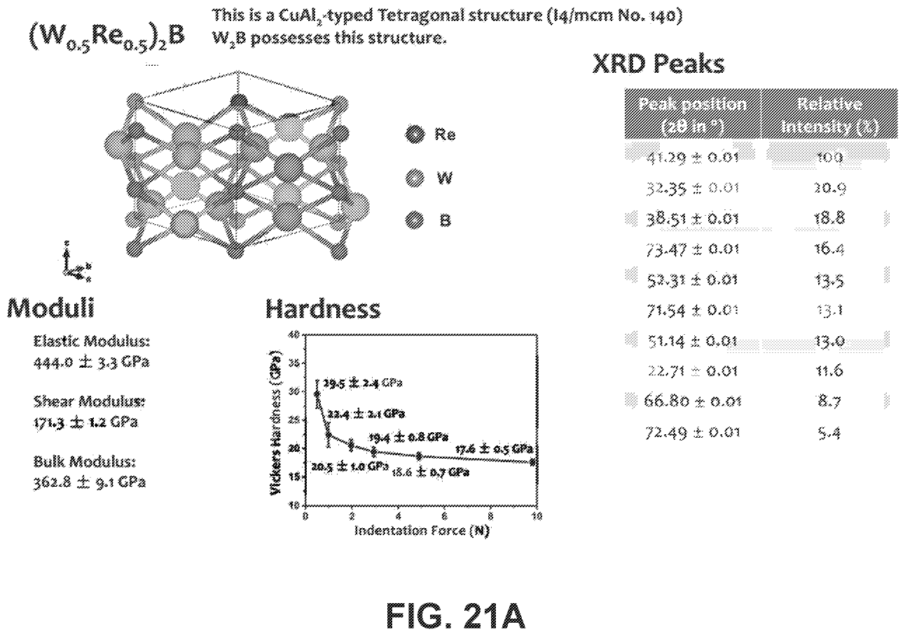

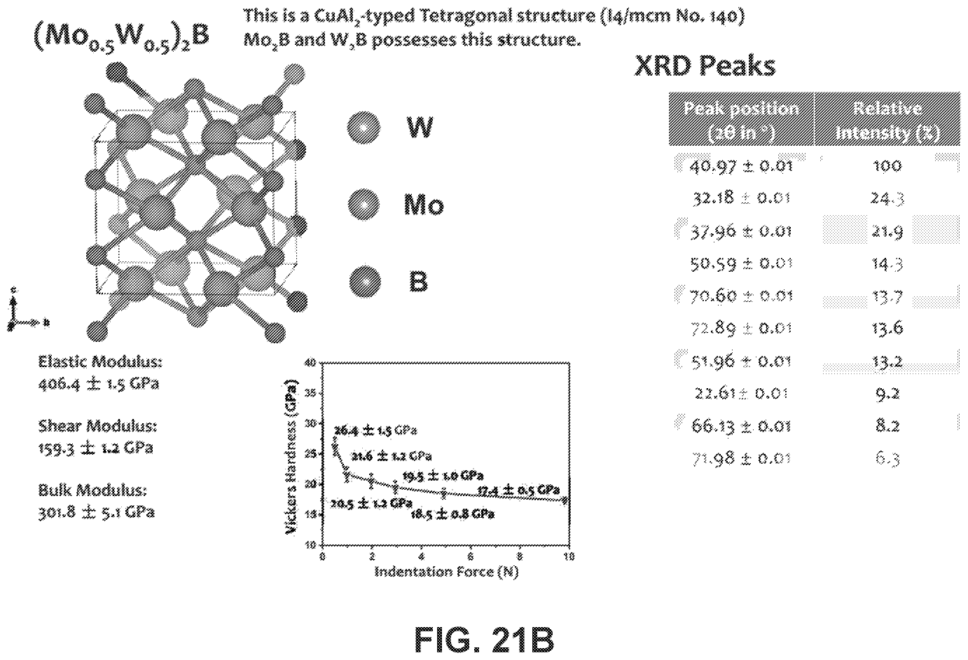

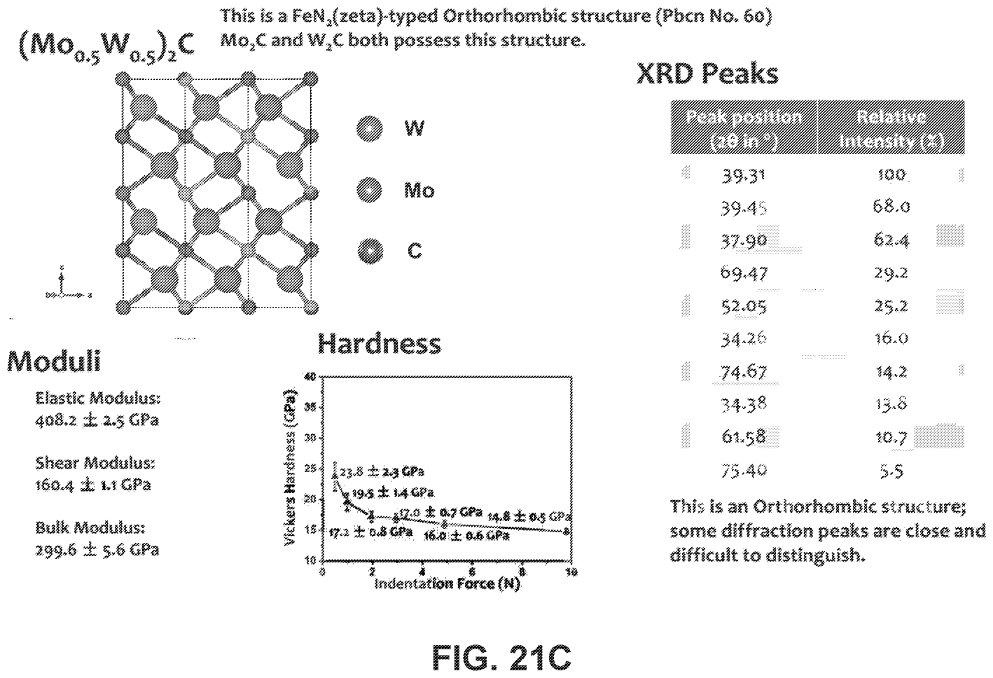

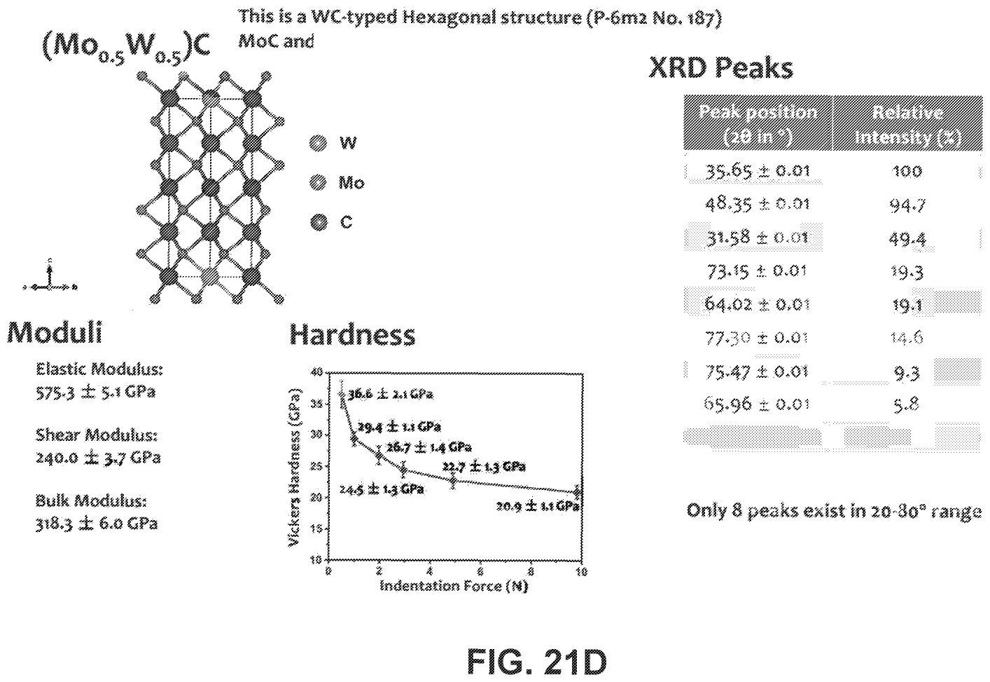

As noted, in some example embodiments, a combination of the Bayesian optimization with symmetry relaxation (BOWSR) algorithm and the machine learning based property prediction model (e.g., the graph based deep learning energy model) was applied to identify a variety of novel ultra-incompressible hard materials, including MoWC₂ (P6₃/mmc) and ReWB (Pca2₁). FIGS. 21A-D depict the characteristics of these novel ultra-incompressible materials. The X-ray power diffraction (XRD) patterns shown in FIGS. 21A-D were collected on a Rigaku Miniflex diffractometer with Cuk Kα radiation at 30 kV and 15 mA under ambient conditions. The scannings were conducted over a 20 range of 20-80°, a step size of 0.01°, at a speed of 2° per minute.

FIG. 21A depicts the characteristics of ReWB, in accordance with some example embodiments. In some example embodiments, the crystalline form of ReWB is characterized by an X-ray powder diffraction pattern comprising peak intensities at 41.29, 32.35, 38.51, 73.47, and 52.31 (±0.1 degrees 2θ). In some example embodiments, the crystalline form of ReWB is characterized by an X-ray powder diffraction pattern comprising peak intensities at 71.54, 51.14, 22.71, 66.80, and 72.49 (±0.1 degrees 2θ).

FIG. 21B depicts the characteristics of (Mo_{0.5}W_{0.5})₂B, in accordance with some example embodiments. In some example embodiments, the crystalline form of (Mo_{0.5}W_{0.5})₂B is characterized by an X-ray powder diffraction pattern comprising peak intensities at 40.97, 32.18, 37.96, 50.59, and 70.60 (±0.1 degrees 2θ). In some example embodiments, the crystalline form of (Mo_{0.5}W_{0.5})₂B is characterized by an X-ray powder diffraction pattern comprising peak intensities at 72.89, 51.96, 22.61, 66.13, and 71.98 (±0.1 degrees 2θ).

FIG. 21C depicts the characteristics of (Mo_{0.5}W_{0.5})₂C, in accordance with some example embodiments. In some example embodiments, the crystalline form of (Mo_{0.5}W_{0.5})₂C is characterized by an X-ray powder diffraction pattern comprising peak intensities at 39.31, 39.45, 37.90, 69.47, and 52.05 (±0.1 degrees 2θ). In some example embodiments, the crystalline form of (Mo_{0.5}W_{0.5})₂C is characterized by an X-ray powder diffraction pattern comprising peak intensities at 34.26, 74.67, 34.38, 61.58, and 75.40 (±0.1 degrees 2θ).

FIG. 21D depicts the characteristics of MoWC₂, in accordance with some example embodiments. In some example embodiments, the crystalline form of MoWC₂ is characterized by an X-ray powder diffraction pattern comprising peak intensities at 35.65, 48.35, 31.58, 73.15, and 64.02 (±0.1 degrees 2θ). In some example embodiments, the crystalline form of MoWC₂ is characterized by an X-ray powder diffraction pattern comprising peak intensities at 77.30, 75.47, and 65.96 (±0.1 degrees 2θ).

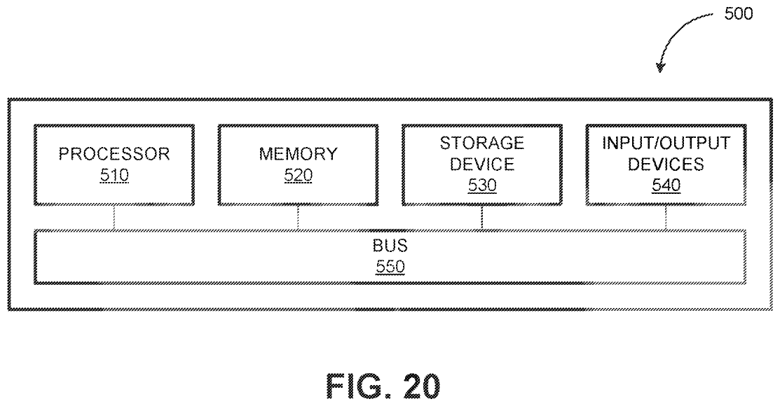

FIG. 20 depicts a block diagram illustrating an example of a computing system 500 consistent with implementations of the current subject matter. Referring to FIGS. 1 and 20, the computing system 500 may implement the design engine 110 and/or any components therein.

As shown in FIG. 20, the computing system 500 can include a processor 510, a memory 520, a storage device 530, and input/output device 540. The processor 510, the memory 520, the storage device 530, and the input/output device 540 can be interconnected via a system bus 550. The processor 510 is capable of processing instructions for execution within the computing system 500. Such executed instructions can implement one or more components of, for example, the design engine 110. In some implementations of the current subject matter, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some implementations of the current subject matter, the input/output device 540 includes a keyboard and/or pointing device. In various implementations, the input/output device 540 includes a display unit for displaying graphical user interfaces.

According to some implementations of the current subject matter, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some implementations of the current subject matter, the computing system 500 can be used to execute various interactive computer software applications that can be used for organization, analysis and/or storage of data in various (e.g., tabular) format (e.g., Microsoft Excel R, and/or any other type of software). Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively, or additionally, store such machine instructions in a transient manner, such as for example, as would a processor cache or other random access memory associated with one or more physical processor cores.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by at least one data processor, cause operations comprising:
applying a first machine learning model trained to generate, based at least on a first crystal structure, an equilibrium crystal structure corresponding the first crystal structure, the first machine learning model generating the equilibrium crystal structure by at least iteratively searching a solution space including a plurality of possible variations of the first crystal structure for a variation of the first crystal structure having a minimum formation energy;
determining, based at least on the equilibrium crystal structure, one or more properties of the first crystal structure; and
identifying, based at least on the one or more properties of the first crystal structure, the first crystal structure as a candidate for synthesis.

2. The system of claim 1, wherein the first machine learning model comprises a Bayesian optimization (BO) model.

3. The system of claim 1, wherein the searching of the solution space is constrained to variations of the first crystal structure having a same symmetry as the first crystal structure.

4. The system of claim 1, wherein the searching of the solution space is constrained based on a symmetry of a lattice of the first crystal structure.

5. The system of claim 1, wherein the searching of the solution space is constrained based on a Wyckoff position of each atom comprising the first crystal structure.

6. The system of claim 1, wherein each variation of the plurality of possible variations of the first crystal structure includes at least one change to a lattice parameter or an atomic coordinate of the first crystal structure.

7. The system of claim 1, wherein the first machine learning model generates the equilibrium crystal structure by at least searching the solution space to identify a first variation of the first crystal structure and determining a first formation energy of the first variation of the first crystal structure.

8. The system of claim 7, wherein the first machine learning model further generates the equilibrium crystal structure by at least searching the solution space to identify a second variation of the first crystal structure, determining a second formation energy of the second variation of the first crystal structure, and in response to the second formation energy being less than the first formation energy, searching, based at least on the second variation of the first crystal structure, the solution space to identify a third variation of the first crystal structure.

9. The system of claim 8, wherein the searching of the solution space includes exploiting an explored portion the solution space by at least identifying a sample having an above-threshold predicted mean as the third variation of the first crystal structure.

10. The system of claim 8, wherein the searching of the solution space includes exploring an unexplored portion of the solution space by at least identifying a sample having an above-threshold predictive uncertainty as the third variation of the first crystal structure.

11. The system of claim 8, wherein the first formation energy and the second formation energy are determined by applying a second machine learning model.

12. The system of any claim 7, wherein the one or more properties are determined by applying a second machine learning model trained to determine the one or more properties.

13. The system of claim 12, wherein the second machine learning model comprises a graph neural network in which atoms in a crystal structure are represented as nodes and bonds in the crystal structure as edges.

14. The system of claim 1, wherein the one or more properties include at least one of a mechanical property, chemical property, thermal property, optical property, or magnetic property of the equilibrium crystal structure.

15. The system of claim 1, wherein the operations further comprise generating, based at least on a second crystal structure, the first crystal structure.

16. The system of claim 15, wherein the first crystal structure is generated by at least substituting a first element of the second crystal structure with a second element.

17. The system of claim 1, wherein the first crystal structure is identified as the candidate for synthesis based at least on the first crystal structure exhibiting an above-threshold elastic modulus.

18. The system of claim 1, wherein the first crystal structure is identified as the candidate for synthesis by in-situ reactive spark plasma sintering.

19. A computer-implemented method, comprising:
applying a first machine learning model trained to generate, based at least on a first crystal structure, an equilibrium crystal structure corresponding the first crystal structure, the first machine learning model generating the equilibrium crystal structure by at least iteratively searching a solution space including a plurality of possible variations of the first crystal structure for a variation of the first crystal structure having a minimum formation energy;
determining, based at least on the equilibrium crystal structure, one or more properties of the first crystal structure; and
identifying, based at least on the one or more properties of the first crystal structure, the first crystal structure as a candidate for synthesis.

20. The method of claim 19, wherein the first machine learning model comprises a Bayesian optimization (BO) model.

21-48. (canceled)
